(19) United States
(12) Patent Application Publication
(10) Pub. No.: US 2021/0171442 A1
(43) Pub. Date: Jun. 10, 2021

(54) PROCESS FOR THE PREPARATION OF BACLOFEN AND ITS INTERMEDIATE

(71) Applicant: PIRAMAL PHARMA LIMITED, Mumbai (IN)

(72) Inventors: Ashutosh JAGTAP, Mumbai (IN); Milind GHARPURE, Mumbai (IN); Navnath SHINDE, Mumbai (IN); Navnath PATIL, Mumbai (IN); Changdev RAUT, Mumbai (IN); Dhileepkumar KRISHNAMURTHY, Mumbai (IN)

(73) Assignee: PIRAMAL PHARMA LIMITED, MUMBAI (IN)

(21) Appl. No.: 17/153,593

(22) Filed: Jan. 20, 2021

Related U.S. Application Data
(62) Division of application No. 15/744,272, filed on Jan. 12, 2018, now abandoned.

(30) Foreign Application Priority Data
Jul. 13, 2015 (IN) 2633/MUM/2015

(51) Int. Cl. C07C 253/30; C07C 227/08

(52) U.S. Cl. CPC C07C 253/30; C07C 255/41; C07C 227/08

(57) ABSTRACT

The present invention provides an improved process for the preparation of 3-(4-chlorophenyl)-3-cyanopropanoic acid (compound (A)) and further its transformation to Baclofen (I). The process comprises reaction of compound (II) with Glyoxylic acid to obtain 3-(4-chlorophenyl)-3-cyanoacrylic acid (III); followed by the ‘in-situ’ reduction of (III) in the presence of a reducing agent to provide the compound (A).

Alternatively, the compound (A) is obtained by the process comprising reacting 2-(4-chlorophenyl)acetonitrile (II) with haloacetic acid (IV) in the presence of a base.

The compound 3-(4-chlorophenyl)-3-cyanopropanoic acid (A) undergoes hydrogenation in the presence of a metal catalyst and ammonia solution to provide Baclofen (I).

### RELATED APPLICATION INFORMATION

This application is a DIVISIONAL of application Ser. No. 15/744,272 filed on Jan. 12, 2018, which is a 371 of International Application of PCT/IB2016/054101 filed on Jul. 8, 2016 and which claims priority from Indian Application No. 2633/MUM/2015 filed on Jul. 13, 2015, the content of which is incorporated herein by reference.

### FIELD OF THE INVENTION

The present invention relates to an improved process for the preparation of 3-(4-chlorophenyl)-3-cyanopropanoic acid (hereinafter referred as the compound (A)), which is useful as a key intermediate for the synthesis of Baclofen (4-amino-3-(p-chlorophenyl) butyric acid). The process of the present invention further involves transformation of the said intermediate cyano compound (the compound (A)) to Baclofen (referred to as the compound (I)) and pharmaceutically acceptable salts thereof.

### BACKGROUND OF THE INVENTION

The following discussion of the prior art is intended to present the invention in an appropriate technical context, and allows its significance to be properly appreciated. Unless clearly indicated to the contrary, reference to any prior art in this specification should not be construed as an expressed or implied admission that such art is widely known or forms part of common general knowledge in the field.

Baclofen (the compound (I)) is a structural analog of the inhibitory neurotransmitter gamma-aminobutyric acid (GABA). The drug is marketed as LIORESAL Intrathecal (Baclofen injection) and is indicated for the management of patients with severe spasticity due to spinal cord injury or multiple sclerosis. Baclofen has the chemical name 4-amino-3-(4-chlorophenyl) butanoic acid and is generally represented as follows:

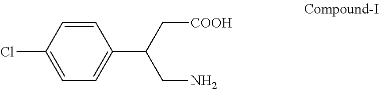 [SMILES: NCC(CC(=O)O)c1ccc(Cl)cc1]

Baclofen being an important drug used in the management of severe spasticity, a number of processes for its preparation as well as for intermediates synthesis are known in the an.

U.S. Pat. No. 3,471,548 describes a process for the synthesis of Baclofen comprises hydrolysis of para-chlorophenyl-glutaric acid imide using sodium hydroxide solution.

U.S. Pat. No. 5,240,925 and J. Org. Chem. Vol 45 (1), pp. 171-73, 1980 describes a process for the synthesis of 2-(4-Chlorophenyl)-3-(3-pyridyl)propionitrile comprises reduction of 2-(4-chlorophenyl)-3-(3-pyridyl) acrylonitrile using sodium borohydride in DMF solvent for atleast 18 hours.

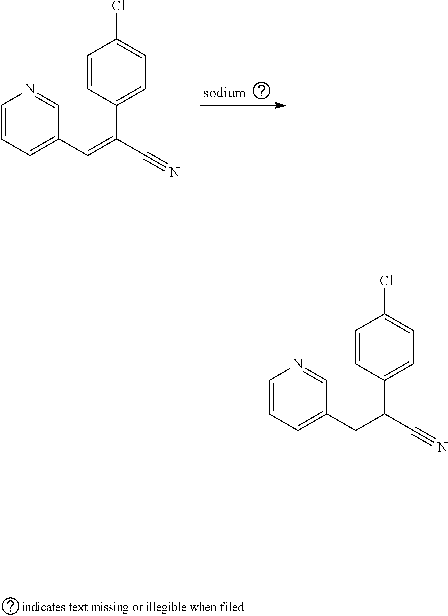 [SMILES: N#C/C(=C/c1cccnc1)c1ccc(Cl)cc1 ; N#CC(Cc1cccnc1)c1ccc(Cl)cc1]

Japanese patent application JP-A3-2006/151839 suggested the hydrogenation of 3-(4-chlorophenyl)-3-cyanopropanoic acid to give 4-amino-3-(4-chlorophenyl)butanoic acid wherein the specification refers that the hydrogenation is carried out under a well-known reducing condition, for example, a Raney nickel catalyst. The said patent application does not provide any working example for the conversion.

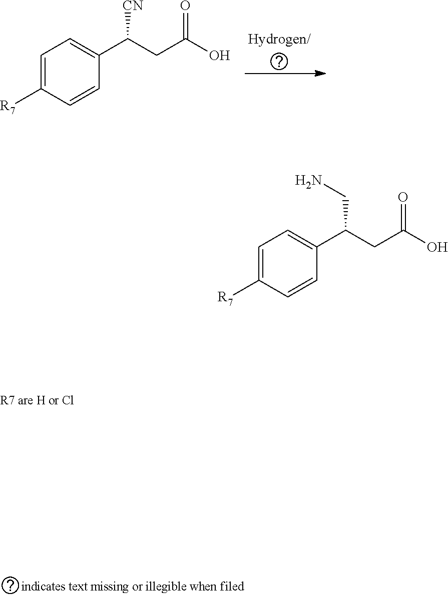

Chinese patent application CN102351726 discloses hydrogenation of 3-(4-chlorophenyl)-3-cyano-propionic acid ethyl ester in the presence of Raney nickel, which gets cyclised immediately to the corresponding pyrrolidone. The product is further treated with an acid solution to give Baclofen hydrochloride.

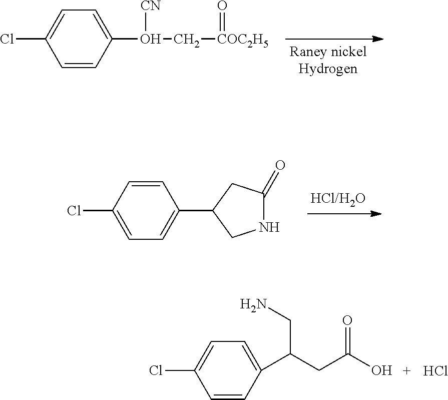

Chinese patent application CN102559553 disclosed asymmetric catalytic reduction of carbon-carbon double bond in the compound 3-(4-chloroaryl)-3-cyano-acrylic acid using Achromobacter sp. JA81 with preservation no. of CCTCC M2011369; the process of CN'553 is depicted below:

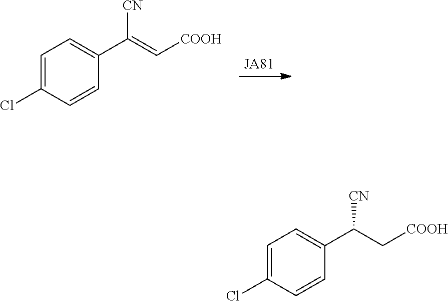 [SMILES: N#C/C(=C\C(=O)O)c1ccc(Cl)cc1 ; N#C[C@H](CC(=O)O)c1ccc(Cl)cc1]

Journal article Synthesis 2001(9) pp 1311-12 disclosed synthesis of arylsuccinic acid comprising alkylation of phenylacetonitrile with salt of chloroacetic acid in the presence of potassium hydroxide in DMF solvent, which finally undergoes hydrolysis.

Similarly. U.S. Pat. No. 5,512,680 describes a process for the synthesis of 3-Cyano-3-(3,4-dichlorophenyl)propionic acid comprising reaction of 3,4-dichlorophenylacetonitrile with dry sodium chloroacetate in the presence of sodium tert-butylate in dry dimethyl sulfoxide for 5 hours.

Various other synthetic methods are disclosed in the U.S. Pat. Nos. 8,273,917; 8,293,926; Chinese patent application CN 101514167 and published PCT application WO-A-2009/044803.

It is evident from the discussion of the processes for the preparation of Baclofen and the intermediate 3-(4-chlorophenyl)-3-cyanopropanic acid (compound-A), described in the afore cited patent documents that some of the reported processes primarily involve critical reaction conditions, prolonged reaction time, use of solvents such as DMF which ends with critical workup procedure, use of complex reagents, purification using column chromatography and expensive solvents; which renders the process costlier and hence the processes are not industrially feasible.

In view of these drawbacks, there is a need to develop an industrially viable commercial process for the preparation of Baclofen and its intermediates: which is a simple, efficient and cost-effective process and provides the desired compounds in improved yield and purity.

Inventors of the present invention have developed an improved process that addresses the problem associated with the processes reported in the prior art. The process of the present invention does not involve use of any toxic and/or costly solvents and reagents. Moreover, the process does not require additional purification steps and critical workup procedure.

Accordingly, the present invention provides a process for the preparation of Baclofen and its intermediates, which is simple, efficient, cost effective, environmentally friendly and commercially scalable for large scale operations.

### SUMMARY OF THE INVENTION

In one aspect, the present invention relates to an improved process for the preparation of 3-(4-chlorophenyl)-3-cyanopropanoic acid (the compound (A)) comprising reacting 2-(4-chlorophenyl)acetonitrile (II) with Glyoxylic acid to obtain 3-(4-chlorophenyl)-3-cyanoacrylic acid (III); followed by the ‘in-situ’ reduction of (I) in the presence of a reducing agent.

In one aspect, the present invention relates to an improved process for the preparation of 3-(4-chlorophenyl)-3-cyanopropanoic acid (the compound (A)) comprising reducing of 3-(4-chlorophenyl)-3-cyanoacrylic acid (II) in the presence of a reducing agent.

In one aspect, the present invention relates to an improved process for the preparation of 3-(4-chlorophenyl)-3-cyanopropanoic acid (the compound (A)) comprising reacting 2-(4-chlorophenyl)acetonitrile (II) with haloacetic acid (IV) in the presence of a base.

In one aspect, the present invention relates to an improved process for the preparation of 3-(4-chlorophenyl)-3-cyanopropanoic acid (the compound (A)) comprising reacting 2-(4-chlorophenyl)acetonitrile (II) with chloroacetic acid in the presence of a base.

In another aspect, the present invention relates to an improved process for the preparation of Baclofen (I) comprising reducing 3-(4-chlorophenyl)-3-cyanopropanoic acid (the compound (A)) in the presence of metal catalyst and ammonia solution.

In another aspect, the present invention relates to an improved process for the preparation of Baclofen (I) comprising reducing 3-(4-chlorophenyl)-3-cyanopropanoic acid (the compound (A)) in the presence of metal catalyst and ammonia solution: wherein the product is treated with EDTA solution to lower the Ni content <30 ppm.

According to another aspect of the present invention, there is provided an improved process for the preparation of Baclofen (I), wherein the said compound (I) has purity of ≥99% with Ni content <30 ppm.

In an another aspect, the present invention relates to an improved process for the preparation of Baclofen (I) comprising, (1) reacting 2-(4-chlorophenyl)acetonitrile (II) with haloacetic acid (IV) in the presence of an base to provide 3-(4-chlorophenyl)-3-cyanopropanoic acid (the compound A); and (2) reducing the compound (A) of stage (1) in the presence of metal catalyst and ammonia solution.

In an another aspect, the present invention relates to an improved process for the preparation of Baclofen (I) comprising, (1a) reacting 2-(4-chlorophenyl)acetonitrile (II) with Glyoxylic acid to obtain 3-(4-chlorophenyl)-3-cyanoacrylic acid (I); followed by the ‘in-situ’ reduction of (I) in the presence of a reducing agent to obtain 3-(4-chlorophenyl)-3-cyanopropanoic acid (the compound A) and; (2a) reducing the compound (A) of stage (1a) in the presence of metal catalyst and ammonia solution.

### DETAILED DESCRIPTION OF THE INVENTION

Accordingly, the present invention relates to an improved process for the preparation of 3-(4-chlorophenyl)-3-cyanopropanoic acid (the compound (A)) represented by the following formula,

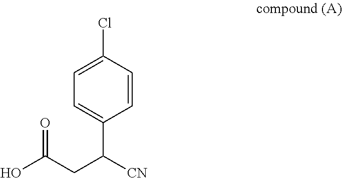 [SMILES: N#CC(CC(=O)O)c1ccc(Cl)cc1]

comprising reducing the compound (III) represented by the following formula

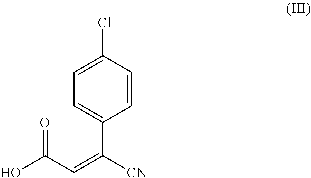 [SMILES: N#C/C(=C/C(=O)O)c1ccc(Cl)cc1]

in the presence of a reducing agent.

The compound (A) obtained by the afore described process is optionally, converted into Baclofen free base or a pharmaceutically acceptable salt thereof.

According to another aspect, the present invention relates to an improved process for the preparation of 3-(4-chlorophenyl)-3-cyanopropanoic acid (the compound (A)) represented by the following formula,

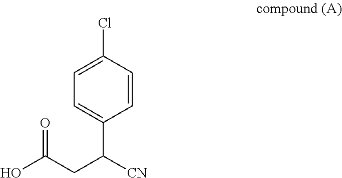 [SMILES: N#CC(CC(=O)O)c1ccc(Cl)cc1]

comprising
(a) reacting the compound (II) represented by the following formula;

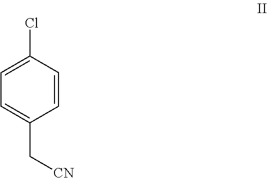

with Glyoxylic acid;
(b) reducing the compound (III) obtained from stage (a) represented by the following formula

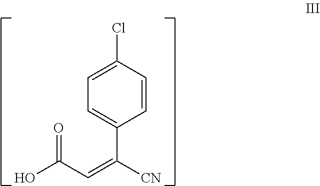

in the presence of a reducing agent;
wherein the reduction at stage (b) is carried out ‘in-situ’.
(c) optionally, converting the compound (A) into Baclofen free base or a pharmaceutically acceptable salt thereof.

In the context of the present invention, the term “optionally” when used in reference to any element; including a process step, e.g. conversion of a compound; it is intended to mean that the subject element is subsequently converted, or alternatively, is not converted to a further compound. Both alternatives are intended to be within the scope of the present invention.

In an embodiment, the reducing agent is selected from the group consisting of hydrides such as sodium borohydride, potassium borohydride, lithium borohydride, zinc borohydride, sodium cyanoborohydride, sodium sulfureted borohydride, sodium trioxyacetal borohydride, sodium tri-alkoxy borohydride, sodium hydroxyl borohydride, sodium borohydride anilide, tetrahydrofuran borohydride, di-methyl-butyl borohydride, lithium-aluminum hydride, lithium-aluminum tri-oxymethyl hydride, sodium-aluminum-2-methoxy-ethoxy hydride, and aluminum hydride and/or mixtures thereof.

In an embodiment, the reducing agent is sodium borohydride.

In a specific embodiment, the process for the preparation of 3-(4-chlorophenyl)-3-cyanopropanoic acid (the compound (A)) comprises the steps of:

(1) dissolving compound (U) in a solvent;
(2) adding glyoxylic acid and abase to the reaction mixture of stage (1);
(3) optionally, filtering the reaction mixture of stage (2);
(4) ‘in-situ’ adding reducing agent to the reaction mixture of stage (3);
(5) stirring the reaction mixture of above step (4) at temperature of about 55° C.:
(6) isolating the desired product;
(7) optionally, converting the product of stage (6) to Baclofen.

The process of the present invention as per the specific embodiment described above is illustrated in the following Scheme-I,

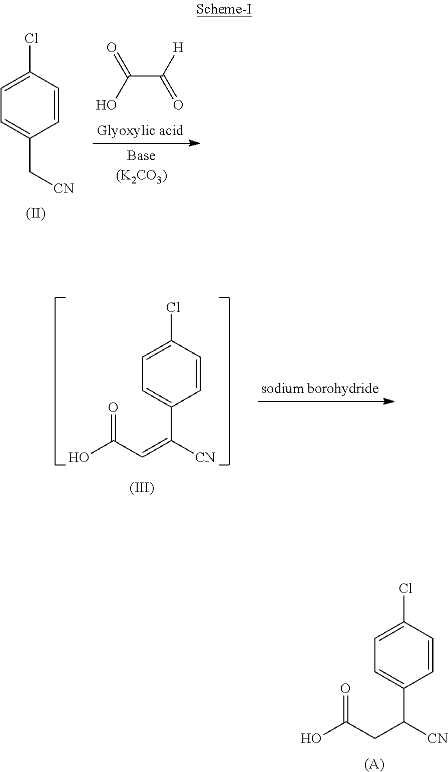

The process as described above further comprises optionally converting the pure compound-A into the Baclofen free base or a pharmaceutically acceptable salt thereof.

The solvent used in the step-(1) of the above process (as depicted in the Scheme-I) is selected from the halogenated solvent such as dichloromethane, 4-bromotoluene, diiodomethane, carbon tetrachloride, chlorobenzene and chloroform; alcoholic solvent such as methanol, ethanol, isopropanol, t-amyl alcohol, t-butyl alcohol and hexanol; ketones such as acetone; an ether solvent such as tetrahydrofuran, cyclopentyl methyl ether, 2-methyltetrahydrofuran, diethyl ether and 1,4-dioxane; an aprotic solvent such as acetonitrile; an aromatic solvent such as toluene, xylene and benzene; water and/or a mixture thereof.

The base used in the step-(2) of the above process is an inorganic base selected from sodium carbonate, potassium carbonate, sodium bicarbonate, cesium carbonate, calcium carbonate, sodium hydroxide or potassium hydroxide.

The reducing agent used in the step-(4) of the above process (as depicted in the Scheme-I) is selected from the group consisting of hydrides such as sodium borohydride potassium borohydride, lithium borohydride, zinc borohydride, sodium cyanoborohydride, sodium sulfurated borohydride, sodium trioxyacetal borohydride, sodium tri-alkoxy borohydride, sodium hydroxyl borohydride, sodium borohydride anilide, tetrahydrofuran borohydride, di-methyl-butyl borohydride, lithium-aluminum hydride, lithium-aluminum tri-oxymethyl hydride, sodium-aluminum-2-methoxy-ethoxy hydride, and aluminum hydride and/or mixtures thereof.

The term ‘temperature of about 55° C.’ referred to in the step (5) of the above process (as depicted in the Scheme I) can range from 45° C. to 65° C. More preferably, the temperature ranges from 50° C. to 60° C.

The term ‘isolating the desired product’ referred to in the step (6) corresponds to the steps involving addition of water, precipitation, separation of solvents, evaporation of solvent, filtration, washing and drying.

The process of the present invention as illustrated in the above Scheme-I comprises addition of glyoxylic acid to the stirring solution of the compound II and potassium carbonate in methanol. The reaction mixture stirred for 3 hours at room temperature and filtered. Aqueous solution of sodium borohydride was added to the methanol solution containing compound-III. The reaction mixture was heated to temperature of about 50-60° C. and continued stirring for 3 hours. The resultant mixture was mixed with water and toluene, and treated with acid solution to provide desired product 3-(4-chlorophenyl)-3-cyanopropanoic acid (the compound A) in a yield of about 90% with purity about 95% (HPLC).

Advantageously, the process of the present invention provides a product with significant improvements in the purity ≥95% and yield upto 90% over the processes reported in the prior art. Also the overall reaction time is also reduced significantly as to 3-6 hours against the reported prior art reaction time of 18-23 hours. Hence, the process of the instant invention effectively contributes to the reduction of overall cost of the process.

Advantageously, the process of the present invention is simpler and it overcomes the drawbacks of the known methods.

Accordingly yet another aspect, the present invention relates to an improved process for the preparation of 3-(4-chlorophenyl)-3-cyanopropanoic acid (the compound (A)) represented by the following formula,

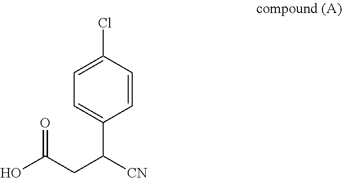 [SMILES: N#CC(CC(=O)O)c1ccc(Cl)cc1]

comprising reacting the compound (II) represented b the following formula;

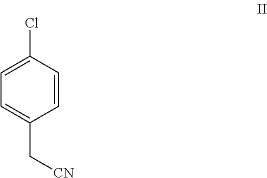

with haloacetic acid (IV) represented by the following formula;

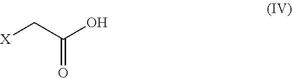

wherein X is halogen selected from F, Cl, Br, I; in the presence of a base.

The compound (A) obtained by the afore described process is optionally, converted into Baclofen free base or a pharmaceutically acceptable salt thereof.

In an embodiment, the base is selected from the group consisting of inorganic base such as sodium carbonate, potassium carbonate, sodium bicarbonate, cesium carbonate, calcium carbonate, sodium hydroxide or potassium hydroxide.

In an embodiment, the base is potassium carbonate.

In another specific embodiment, the process for the preparation of 3-(4-chlorophenyl)-3-cyanopropanoic acid (the compound (A)) comprises the steps of:

(i) dissolving haloacetic acid (IV) in a solvent;
(ii) adding a base to the stirring solution of stage (i);
(iii) cooling the reaction mixture of stage (ii) to a temperature of about 10° C.;
(iv) adding compound (II) to the stirring solution of stage (iii);
(v) adding a base to the stirring solution of stage (iv);
(vi) isolating the desired product; and
(vii) optionally, converting the product of stage (v) to Baclofen.

The process of the present invention as per the specific embodiment described above is illustrated in the following Scheme-II,

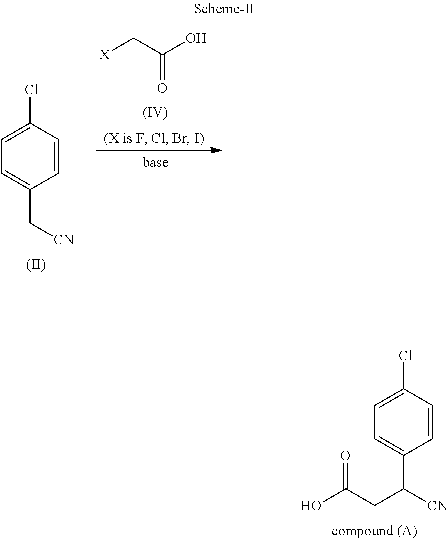

The process as described above further comprises optionally converting the pure compound-A into the Baclofen free base or a pharmaceutically acceptable salt thereof.

The solvent used in the step-(i) of the above process (as depicted in the Scheme-I) is selected from the halogenated solvent such as dichloromethane, 4-bromotoluene, diiodomethane, carbon tetrachloride, chlorobenzene and chloroform alcoholic solvent such as methanol, ethanol, isopropanol, t-amyl alcohol, t-butyl alcohol and hexanol; ketones such as acetone; an ether solvent such as tetrahydrofuran, cyclopentyl methyl ether, 2-methyltetrahydrofuran, diethyl ether and 1,4-dioxane; an aprotic solvent such as acetonitrile; an aromatic solvent such as toluene, xylene and benzene, dimethyl sulfoxide (DMSO); water and/or a mixture thereof.

The base used in the step-(ii) and step-(v) of the above process is an inorganic base selected from sodium carbonate, potassium carbonate, sodium bicarbonate, cesium carbonate, calcium carbonate, sodium hydroxide or potassium hydroxide.

The term ‘temperature of about 10° C.’ referred to in the step (i) of the above process (as depicted in the Scheme II) can range from 0° C. to 20° C. More preferably, the temperature ranges from 5° C. to 15° C.

The term ‘isolating the desired product’ referred to in the step (vi) corresponds to the steps involving addition of water, precipitation, separation of solvents, evaporation of solvent, filtration, washing and drying.

The process of the present invention as illustrated in the above Scheme-I comprises addition of chloroacetic acid to the stirring solution of the compound (H) and potassium carbonate in DMSO. The reaction mixture was cooled to the temperature of about 10° C., and was added compound-H and potassium hydroxide. The reaction mixture was stirred for 1.5 hours and organic layers were separated by the addition of water and toluene. The organic layer was treated with acid solution and further with ammonia to provide desired product 3-(4-chlorophenyl)-3-cyanopropanoic acid (the compound A) in a yield of about 90% with a purity about 95% (HPLC).

Advantageously, the process of present invention according to scheme U provides a product with significant improvements in the purity about 95% and yield upto 90% over the processes reported in the prior art. Also the overall reaction time is also reduced significantly to 1-2 hours against the reported prior art reaction time of 5-6 hours. Hence, the process of the instant invention effectively contributes to the reduction of overall cost of the process.

According to yet another aspect, the present invention relates to an improved process for the preparation of Baclofen (compound-I) represented by the following formula.

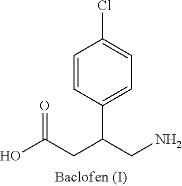 [SMILES: NCC(CC(=O)O)c1ccc(Cl)cc1]

comprising reducing the compound (A) represented by the following formula;

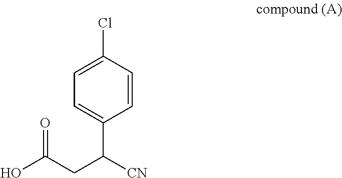 [SMILES: N#CC(CC(=O)O)c1ccc(Cl)cc1]

in the presence of metal catalyst and ammonia solution.

In an embodiment, the metal catalyst is selected from the group consisting of Nickel. Raney Nickel, palladium, platinum, zinc, iron (Fe) and Sn.

In an embodiment, the metal catalyst is Raney Nickel.

In an embodiment, the ammonia solution is selected from the group consisting of aqueous ammonia and/or alcoholic ammonia such as methanolic ammonia, ammonia in isopropyl alcohol (IPA-ammonia).

In another specific embodiment, the process for the preparation of Baclofen (I) comprises the steps of:

(m) dissolving compound (A) in an ammonia solution:
(n) adding metal catalyst to the stirring solution of stage (m):
(o) applying the hydrogen pressure to the reaction mixture of stage (n);
(p) stirring the reaction mixture of stage (o) at room temperature;
(q) isolating the desired product.
(r) optionally, treating the product with EDTA solution.

The process of the present invention as per the specific embodiment described above is illustrated in the following Scheme-II.

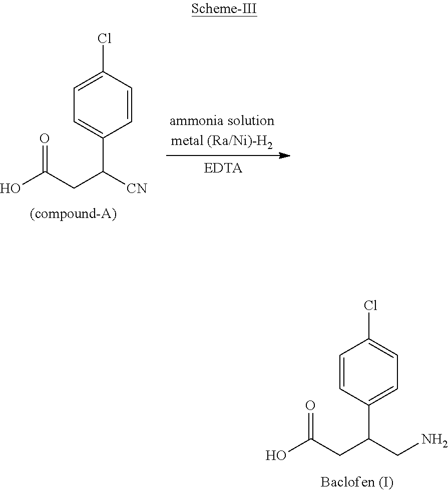

The ammonia solution used in the step-(m) of the above process (as depicted in the Scheme-I) is selected from aqueous ammonia alcoholic ammonia such as methanolic ammonia or IPA ammonia.

The metal catalyst used in the step-(n) of the above process (as depicted in the Scheme-III) is selected from the group consisting of Nickel, Raney Nickel, palladium, platinum, zinc, iron (Fe) and Sn.

The term ‘room temperature’ referred to in the step (p) of the above process (as depicted in the Scheme III) can range from 25° C. to 30° C.

The term ‘isolating the desired product’ referred to in the step (q) corresponds to the steps involving addition of water, precipitation, separation of solvents, evaporation of solvent, filtration, washing and drying.

The process of the present invention as illustrated in the above Scheme-III comprises addition of compound-A to the aqueous ammonia solution at room temperature. Raney nickel was added to the said reaction mixture and stirred for 4 hours under hydrogen pressure at room temperature. The ammonia was distilled off and the reaction mixture was treated with EDTA disodium salt solution, to obtain desired product (Baclofen) in a yield of about 70% with a purity of about ≥99% (HPLC), with a Ni content <30 ppm.

In an embodiment, the final product is treated with EDTA solution to obtain a product with lower Nickel content. The EDTA solution is EDTA disodium salt solution; which is prepared by adding EDTA disodium salt in water into 50% aqueous sodium hydroxide solution till a clear solution results. Advantageously, the process of present invention provides product with ‘Ni’ content <30 ppm.

Advantageously, the process of present invention according to Scheme II provides a product with significant improvements in the purity about ≥99% and yield upto 70% over the processes reported in the prior art. Also the overall reaction time is also reduced significantly as to 7-9 hours against the reported prior art reaction time of 15 hours. Eventually, the process of instant invention effectively contributes to the reduction of overall cost of the process. Hence, the process of present invention is simpler and it overcomes the drawbacks of the known methods.

According to the above embodiments, the Baclofen (I) is obtained by the process as depicted in the following Scheme-IV;

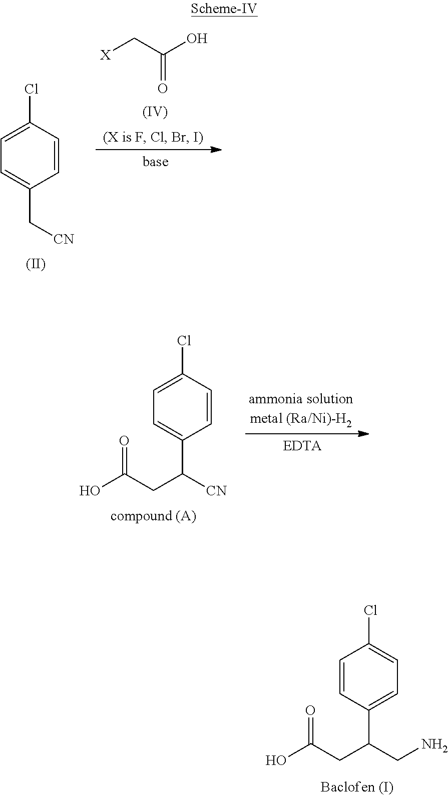

The process of scheme-IV comprises the reaction of 2-(4-chlorophenyl)acetonitrile (II) with haloacetic acid (IV) in the presence of an base as defined earlier to provide 3-(4-chlorophenyl)-3-cyanopropanoic acid (A). The compound (A) further reduced in the presence of metal catalyst as defined earlier and ammonia solution to provide Baclofen (I).

According to the above embodiments, the Baclofen (I) is obtained by the process as depicted in the following Scheme-V:

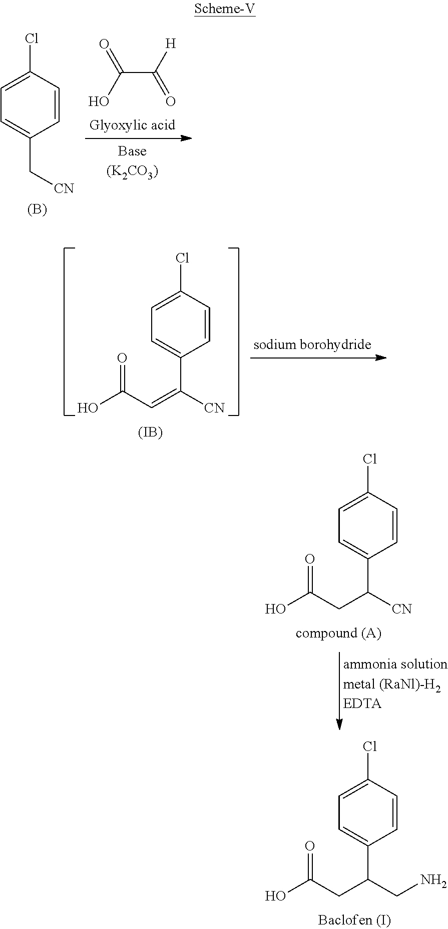

The process of scheme-V comprises the reaction of 2-(4-chlorophenyl)acetonitrile (II) with Glyoxylic acid to obtain 3-(4-chlorophenyl)-3-cyanoacrylic acid (I); followed by the ‘in-situ’ reduction of (III) in the presence of a reducing agent as defined earlier to obtain 3-(4-chlorophenyl)-3-cyanopropanoic acid (the compound A). The compound (A) is further reduced in the presence of metal catalyst as defined earlier and ammonia solution to provide Baclofen (I).

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Example-1: Preparation of 3-(4-chlorophenyl)-3-cyanopropanoic acid (A)

Charged dimethyl sulfoxide (1750 ml) in a flask followed by the addition of chloroacetic acid (494 g, 0.95 meq.) and potassium carbonate powder (550 g, 1.2 meq.). To the stirring solution was added 2-(4-chlorophenyl) acetonitrile (II)(500 g, 1.0 meq.) and potassium hydroxide powder (323 g, 1.7 meq.), the reaction mixture was stirred for 90 min at a temperature of about 10-20° C. The reaction mixture was quenched by adding water (2500 mL) and toluene (1000 mL). The separated aqueous layer was acidified to a pH of 2.0 using concentrated hydrochloric acid (1050 mL) and the product was extracted in toluene (2000 ml). The organic layer was treated with aqueous ammonia. The separated aqueous layer was treated with concentrated hydrochloric acid (1000 mL) and stirred at temperature about 10-20° C. for 30 min. The desired precipitated product was isolated by filtration with a yield of 90% and purity of 95% (HPLC).

### Example-2: Preparation of 3-(4-chlorophenyl)-3-cyanopropanoic acid (A)

Charged methanol (1400 mL) in flask followed by the addition of 2-(4-chlorophenyl)acetonitrile (II)(200 g, 1.0 meq.), potassium carbonate powder (420 g, 2.3 meq.) and Glyoxylic acid (146.5 g, 1.5 meq.). The reaction mixture was stirred at temperature about 25-30° C. for 3 hours. To the reaction mixture was added water (100 mL) and the precipitated product (E)-3-(4-chlorophenyl)-3-cyanoacrylic acid (III) was isolated by filtration. To the wet solid was added methanol (1800 mL) and sodium borohydride (67 g, 1.3 meq.). The reaction mixture was stirred at temperature about 50-60° C. for 3 hours. Methanol was distilled off, to the residue was added water (3 V) and toluene (2 V). The separated aqueous layer was treated with concentrated hydrochloric acid (300 mL) to pH 1-1.5) and the desired precipitated product was isolated by filtration with a yield of 80% and purity of 95% (HPLC).

### Example 3: Preparation of Baclofen (I)

Charged compound 3-(4-chlorophenyl)-3-cyanopropanoic acid (A)(100 g) and aqueous ammonia (600 mL) in a hydrogenation reactor, followed by the addition of Raney nickel (10 g). The reaction mixture was stirred under hydrogen pressure (10 kg) at temperature about 25-30° C. for 5 hours. The reaction mixture was filtered and the excess ammonia was distilled off.

The aqueous layer was treated with 50% sodium hydroxide solution (50 mL) and was further treated with ethylenediaminetetra acetic acid disodium salt dihydrate (5 g). The aqueous layer was extracted with MDC (50 mL) and the separated aqueous layer was acidified using dilute hydrochloric acid solution (pH 6.5-7) followed by the addition of iso-propanol (200 mL). The desired precipitated product was isolated by filtration with a yield of 70% and a purity of 99% (HPLC).

1. A process for the preparation of 3-(4-chlorophenyl)-3-cyanopropanoic acid (A) of the following formula, 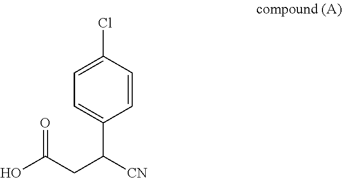 [SMILES: N#CC(CC(=O)O)c1ccc(Cl)cc1]
comprising, reacting the compound (II) of the following formula; 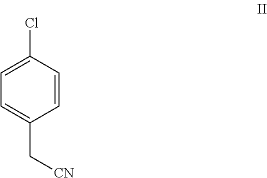
with haloacetic acid (IV) of the following formula; 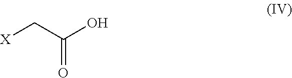
wherein X is halogen selected from F, Cl, Br, and I; in the presence of a base.

2. The process according to claim 1, wherein the base is selected from the group consisting of sodium carbonate, potassium carbonate, sodium bicarbonate, cesium carbonate, calcium carbonate, sodium hydroxide and/or potassium hydroxide.

3. (canceled)

4. A process for the preparation of 3-(4-chlorophenyl)-3-cyanopropanoic acid (A) of the following formula, 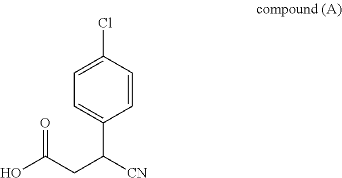 [SMILES: N#CC(CC(=O)O)c1ccc(Cl)cc1]
comprising the steps of,
(a) reacting the compound (II) of the following formula; 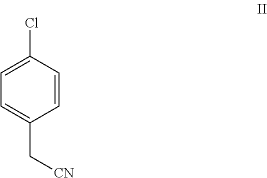
with Glyoxylic acid of the following formula; 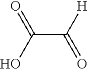
(b) reducing the compound (III) obtained from stage (a) of the following formula 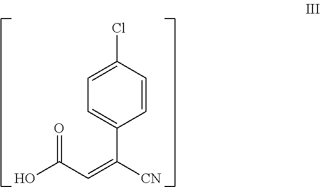
in the presence of a reducing agent; wherein the reduction at stage (b) carried out ‘in-situ’.

5. The process according to claim 4, wherein the reducing agent is selected from the group consisting of sodium borohydride, potassium borohydride, lithium borohydride, zinc borohydride, sodium cyanoborohydride, sodium sulfurated borohydride, sodium trioxyacetal borohydride, sodium tri-alkoxy borohydride, sodium hydroxyl borohydride, sodium borohydride anilide, tetrahydrofuran borohydride, di-methyl-butyl borohydride, lithium-aluminum hydride, lithium-aluminum tri-oxymethyl hydride, sodium-aluminum-2-methoxy-ethoxy hydride, and aluminum hydride and/or mixtures thereof.

6. (canceled)

7. A process for the preparation of Baclofen (I) of the following formula, 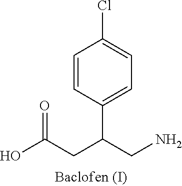 [SMILES: NCC(CC(=O)O)c1ccc(Cl)cc1]
comprising reducing the compound (A) represented by the following formula; 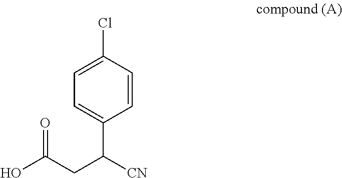 [SMILES: N#CC(CC(=O)O)c1ccc(Cl)cc1]
in the presence of a metal catalyst and ammonia solution.

8. The process according to claim 7, wherein the metal catalyst is selected from the group consisting of Nickel, Raney Nickel, palladium, platinum, zinc, iron (Fe) and tin (Sn).

9. The process according to claim 7, wherein the ammonia solution is selected from the group consisting of aqueous ammonia and/or alcoholic ammonia.

10. The process according to claim 7, wherein the reduction is carried out in the presence of hydrogen source or hydrogen gas.

11. A process for the preparation of Baclofen (I) of the following formula 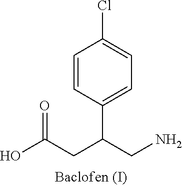 [SMILES: NCC(CC(=O)O)c1ccc(Cl)cc1]
wherein compound (A) prepared according to claim 1 is reduced 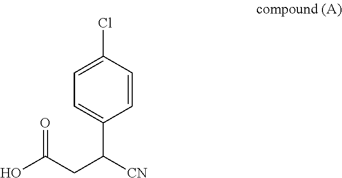 [SMILES: N#CC(CC(=O)O)c1ccc(Cl)cc1]
in the presence of metal catalyst and ammonia solution.

12. (canceled)

13. A process for the preparation of Baclofen (I) of the following formula, 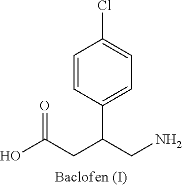 [SMILES: NCC(CC(=O)O)c1ccc(Cl)cc1]
wherein compound (A) prepared according to claim 4 is reduced; 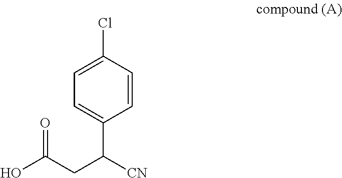 [SMILES: N#CC(CC(=O)O)c1ccc(Cl)cc1]
in the presence of metal catalyst and ammonia solution.

14. (canceled)

15. The process according to claim 13, wherein the metal catalyst is selected from the group consisting of Nickel, Raney Nickel, palladium, platinum, zinc, iron (Fe) or Sn.

16. The process according to claim 13, wherein the ammonia solution is selected from the group consisting of aqueous ammonia and/or alcoholic ammonia.

17. The process according to claim 13, wherein the reduction is carried out in the presence of hydrogen source or hydrogen gas.

18. (canceled)

19. A process for obtaining Baclofen (I) with Nickel (Ni) content less than 30 ppm; comprising treating the compound with EDTA solution.

20. The process according to claim 19, for obtaining Baclofen (I) with Ni content less than 30 ppm; comprising reducing 3-(4-chlorophenyl)-3-cyanopropanoic acid (A) in the presence of metal catalyst and ammonia solution; and treating the product with EDTA solution.

21. The process according to claim 19, wherein the EDTA solution is EDTA disodium salt solution.

22. The process according to claim 11, wherein the metal catalyst is selected from the group consisting of Nickel, Raney Nickel, palladium, platinum, zinc, iron (Fe) or Sn.

23. The process according to claim 11, wherein the ammonia solution is selected from the group consisting of aqueous ammonia and/or alcoholic ammonia.

24. The process according to the claim 11, wherein the reduction is carried out in the presence of hydrogen source or hydrogen gas.

25. The process according to claim 20, wherein the EDTA solution is EDTA disodium salt solution.
